(19) United States
(12) Patent Application Publication
(10) Pub. No.: US 2025/0345273 A1
(43) Pub. Date: Nov. 13, 2025

(54) DEGRADABLE POLY(VINYL ALCOHOL) HYDROGELS FOR WOUND HEALING APPLICATIONS

(71) Applicants: May Beth Monroe, Syracuse, NY (US); Nghia Le Ba Thai, Syracuse, NY (US)

(72) Inventors: May Beth Monroe, Syracuse, NY (US); Nghia Le Ba Thai, Syracuse, NY (US)

(73) Assignee: Office of Technology Transfer, Syracuse, NY (US)

(21) Appl. No.: 19/206,138

(22) Filed: May 13, 2025

Related U.S. Application Data
(60) Provisional application No. 63/646,036, filed on May 13, 2024.

(51) Int. Cl. A61K 9/06; A61K 35/28; A61K 47/32; A61K 47/42; A61P 17/02

(52) U.S. Cl. CPC A61K 9/06; A61K 35/28; A61K 47/32; A61K 47/42; A61P 17/02

(57) ABSTRACT

A tunable hydrogel platform with the capability to encapsulate cells to enhance wound treatment, particularly in chronic wounds or in Crohn's fistulas. Poly(vinyl alcohol) (PVA) was functionalized with thiol and methacrylate groups to enable crosslinking into a degradable 3D hydrogel. Methacrylated gelatin (GelMA) may be added into the hydrogel to improve cell attachment, and porous structures can be introduced via a gas-blowing process to improve nutrient transfer and waste removal and further support cell viability.

### BACKGROUND OF THE INVENTION

### 1. Field

The present disclosure relates to wound dressings and, more specifically, to a tunable polyvinyl alcohol hydrogel for encapsulation of cells to enhance wound treatment.

### 2. Description of the Related Art

Chronic wounds, defined as those failing to restore tissue integrity and functionality within 3 months, pose a significant healthcare challenge. In 2014, the treatment costs for nonhealing chronic wounds were estimated to range between $28 and $97 billion. These wounds are particularly difficult to manage due to their complex pathophysiology, which involves proinflammatory mechanisms, elevated levels of reactive oxygen species (ROS), proteases, bacterial infection, and dysfunctional stem cells.

Current chronic wound treatments include debridement and infection control, negative pressure wound therapy, cell and growth factor delivery, extracellular matrices (ECMs), and skin substitution. Despite extensive research and clinical efforts at improving chronic wound outcomes, the pathology and irregular structure of chronic wounds present significant treatment challenges due to limited accessibility and treatment options.

Tissue engineering approaches have been applied to treat irregularly shaped wounds, as they act like scaffolds for tissue formation and biochemical molecular transportation. However, conventional tissue engineered products often fail to address these types of wounds due to adherence issues. While ECM scaffolds derived from animals can be used, they may not be suitable for deeper, tunneling wounds due to the challenges in controlling scaffold degradation rate to match wound closure rates.

Cell delivery methods have shown potential in chronic wound treatment, especially mesenchymal stem cells (MSCs), which are multifunctional in terms of their differentiation and immunomodulatory properties. In prior work, MSCs demonstrate advanced tissue regeneration, promotion of angiogenesis, and anti-inflammatory properties. Different strategies have been applied to deliver MSCs into wounded areas, including subcutaneous injections and scaffolds loaded with cells. In the injection method, MSCs secrete several paracrine factors, such as angiopoietin 1 and vascular endothelial growth factor, which are important for wound healing. MSCs could also differentiate into various types of cells in wounded areas to promote wound closure. However, injecting MSCs into the wound bed in a soluble form may not be suitable due to low MSC engraftment in the local wound area that can reduce the potential healing capacity of stem cells. In particular, treating deep and/or irregularly shaped wounds requires a delivery system capable of localized stem cell retention to maximize effectiveness.

MSCs can be loaded into biocompatible scaffolds that support cell attachment and function while protecting the cells from the inflammatory wound environment. For example, bioprinting with molding has been utilized to assist hydrogel-loaded cells to treat irregularly shaped wounds, but this process can be time consuming and complicated to scale-up. Electrospun fibers have been loaded with MSCs for application in wound treatment. However, the 2D fiber mats are not capable of thoroughly filling deeper wounds and maintaining high concentrations of MSCs during treatment. Thus, there remain major challenges in fabrication of scaffolds for MSC delivery to irregularly shaped wounds.

Hydrogels have gained significant attention as three-dimensional scaffolds for wound healing due to their distinctive features. They provide a moist environment and mimic the ECM, reduce tissue necrosis, and promote epithelium formation. Hydrogels with unique mechanical properties can be designed with control over cell encapsulation and bioactive agent release. Flexible and degradable hydrogels that can potentially fill irregular wounds support delivered MSCs in releasing cytokines and interacting with the local microenvironment to facilitate wound closure.

As a result, there is a need in the art for a polymer composition that can be readily and inexpensively formed into hydrogel foams that encapsulate cells for use as wound dressings.

### BRIEF SUMMARY OF THE INVENTION

The present invention provides a tunable hydrogel platform with the capability to encapsulate cells to enhance wound treatment, particularly in chronic wounds or in Crohn's fistulas. Poly (vinyl alcohol) (PVA) functionalized with thiol or methacrylate groups was synthesized to enable crosslinking into a degradable 3D hydrogel system. Methacrylated gelatin (GelMA) may be added into the system to improve cell attachment, and porous structures can be introduced via a gas-blowing process to improve nutrient transfer and waste removal and further support cell viability. When the hydrogel was modified using a cytocompatible gas blowing process, porous hydrogel foams with regularly distributed encapsulated MSCs were enabled within minutes. This approach of the present invention has tunable properties to enable long-term MSC viability during chronic wound treatment with high potential for facile scale-up of fabrication processes.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is an illustration of the synthesis of a thiolated polyvinyl alcohol (PVA)/PVA methacrylate/gelatin methacrylate hydrogel system based on thiol-ene reactions between thiolated PVA and PVA methacrylate and/or gelatin methacrylate according to the present invention.

FIG. 2 is a series of graphs showing the chemical characterization of synthesized materials via Fourier transform infrared (FTIR) and 1H-nuclear magnetic resonance (NMR) spectroscopy: (A) FTIR and (B) NMR spectra of thiolated poly(vinyl alcohol); (C) FTIR and (D) NMR spectra of poly(vinyl alcohol) methacrylate (PVAMA); and (E) FTIR and (F) NMR spectra of gelatin methacrylate (GelMA).

FIG. 3 is a series of images and graphs of the chemical and physical characterization of fabricated hydrogels: (A) Image of hydrogels after fabrication; (B) FTIR spectra of hydrogel samples after crosslinking; and (C) FTIR spectra to characterize thiol functional group in TPVA-containing crosslinked hydrogels.

FIG. 4 is a series of images and graphs showing the mechanical and structural property characterization of fabricated hydrogels: (A) Gel fraction; (B) swelling ratio; (C) compressive modulus; (D) macroscale and microscale images of PVA14+GelMA samples fabricated without pores; (E) Macroscale and microscale images of PVA14+GelMA samples fabricated with pores (scale bars apply to all SEM images); (F) Oxidative degradation in 3% H2O2; (G) “Real-time” hydrolytic degradation in PBS; and (H) accelerated hydrolytic degradation in 0.1 M NaOH. n=3, mean±standard deviation displayed. *p<0.05. #Loss of sample integrity, preventing further measurement; xcomplete dissolution.

FIG. 5 is a series of graphs showing the characterization of PVA14 hydrogel cytocompatibility and encapsulated cell viability within PVA14 hydrogels: (A) Representative images of NIH/3T3 fibroblast cytocompatibility in presence of hydrogel library determined using live/dead assay, scale bar=100 μm applies to all images; (B) 3T3 viability data of hydrogel samples, n=5, mean±standard deviation displayed; (C) Illustration of tested sample planes within PVA14 hydrogels (T: Top, M: Middle, B: Bottom); Encapsulated A375 epithelial cell viability at (D) 7 and (E) 14 days within varied planes of hydrogel samples. n=5, mean±standard deviation displayed, *p<0.05.

FIG. 6 is a series of graphs showing the characterization of encapsulated 3T3 cell proliferation and morphology PVA14 hydrogels. Images of encapsulated 3T3/GFP cells in hydrogels (A) in 3D directly after encapsulation (scale bar=500 μm applies to all images); (B) in 2D over 14 days (scale bar=100 μm applies to all images); (C) in scanning electron micrographs at 14 days; and Quantified 3T3/GFP (D) density and (E) spreading over time during encapsulation within hydrogels. n=3, mean±standard deviation displayed, *p<0.05.

FIG. 7 is a series of graphs of the effects of mesenchymal stem cell (MSC) encapsulation in PVA14 hydrogels on wound healing in vitro and on MSC markers: (A) Images of 3T3/GFP fibroblasts at 0 and 24 h of the 2D in vitro scratch wound healing assay. Scale bar applies to all images. Overlaid quantification of wound area closure is presented as mean±standard deviation; (B) Quantified wound area closure at 0 and 24 h with schematic representation of assay setup. n=3, mean±standard deviation displayed, *p<0.05; and (C) Immunofluorescence staining of MSCs encapsulated in PVA14 (+G/+P) with Actin/CD90/DAPI (scale bar=100 μm and applies to all images).

FIG. 8 is a series of images and graphs of the Effects of hydrogels on wound healing ex vivo: (A) Histology images of ex vivo porcine skin closure assay at 3 days; Inserts show epidermis area used for analysis (˜50 μm from the surface); White arrows indicate active cells (identified by Ki67/DAPI) that were counted in quantitative analysis. Scale bar applies to all images; (B) Quantified active cell density at wound edge. n=3 mean±standard deviation displayed, *p<0.05; (C) Quantified encapsulated cell viability during ex vivo assay. n=3 mean±standard deviation displayed.

### DETAILED DESCRIPTION OF THE INVENTION

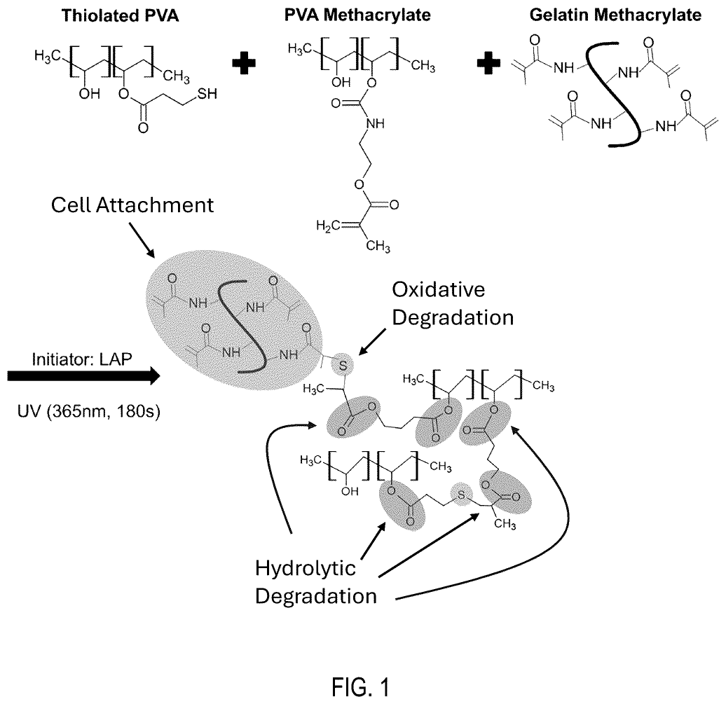

Referring to the figures, wherein like numerals refer to like parts throughout, there is seen in FIG. 1 an approach for the synthesis of a thiolated polyvinyl alcohol (PVA)/PVA methacrylate/gelatin methacrylate hydrogel system that combines TPVA, PVAMA, and GelMA through a thiol-ene reaction. The present invention provides a versatile hydrogel system for chronic wound treatment that uses a cytocompatible gas blowing process to enable porous hydrogel foams with regularly distributed encapsulated MSCs within minutes. This system of the present invention has tunable properties to enable long-term MSC viability during chronic wound treatment with high potential for facile scale-up of fabrication processes. The present invention thus allows for the synthesis of hydrogels with tunable chemistries and porosities that provide a tunable hydrogel foam system with high-encapsulated cell viability and functionality for chronic wound treatment.

### EXAMPLE

### Materials

All reagents and chemicals were purchased from ThermoFisher (Waltham, MA) and used as received unless otherwise specified.

### Synthesis

To obtain thiolated PVA (TPVA), the synthesis was based on a previously described esterification of PVA with hydrochloric acid (HCl). In brief, 10 g of PVA (25,000g/mol) was dissolved in 40 ml of deionized water at 80° C. for 1 h. A combination of 12 g of 3-mercaptopropionic acid and 2 ml HCl (7N, 7 mmol) was added dropwise and allowed to react for 3 hr. The solution was precipitated in acetone twice to obtain the TPVA. TPVA was air dried for 24 h, and then dried under vacuum at room temperature for 24 h before storing in the freezer until further use.

For PVA methacrylate (PVAMA), the modification process was carried out as previously described. Briefly, 5 g of PVA (25,000 g/mol, Polysciences, Warrington, PA) was dissolved in 100 ml of dimethyl sulfoxide (DMSO) at 90° C. for 1 hr. Then, 0.7 g of 2-isocyanatoethyl methacrylate was added dropwise and allowed to react for 4 hr. The product was precipitated in cold acetone to obtain PVAMA. To remove DMSO, the product was then dissolved in phosphate buffered saline (PBS, 10% w/v) at 90° C. for 1 hr and then precipitated again in cold acetone to obtain the final PVAMA product. PVAMA was dried for 24 hr, using the same process used for TPVA.

To synthesize gelatin methacrylate (GelMA), 10 g of type A porcine gelatin was completely dissolved in 98.75 ml of PBS at 60° C. Then, 1.25 ml of methacrylic anhydride (1.25% v/v) was added to the solution at 0.5 ml/min and reacted at 50° C. for 1 hr. To stop the reaction, 2× volume of PBS warmed to 40° C. was added to the solution. The solution was dialyzed using 3.5 kDa MWCO cutoff dialysis tubing for 3 days to remove salts and methacrylic acid. The solution was then lyophilized for 3 days and then stored at −80° C. for further use.

### Spectroscopic Analysis

Attenuated total reflectance-Fourier transform infrared (ATR-FTIR) and 1H nuclear magnetic resonance (NMR) spectroscopy were utilized to validate the structures of TPVA, PVAMA, and GelMA. Dried samples were scanned between 4000 and 400 cm−1 at a resolution of 4 cm−1 using a iD7 ATR-FTIR (Thermo Scientific, Waltham, MA).

For NMR, samples were dissolved in D20, and spectra were obtained using an Advance III HD 400 MHz NMR (Bruker). The results were used to calculate the relatively degree of substitution (DS). For TPVA, the DS was measured based on the ratio of the area under the 3-mercaptopropionic ester proton peak (δ=2.7-2.8 ppm) to the area under the main chain proton peaks (δ=1.4-1.9 and 3.8-4.1 ppm). For PVAMA, the DS was calculated based on the ratio of the area under the methacrylate vinyl proton peaks (δ=6.1 and 5.8 ppm) to the area under the proton peak associated with the PVA backbone (δ=4.0 ppm). For GelMA, the DS was acquired using the area under the methacrylate double bond protons peaks (δ=5.6 and 5.8 ppm) peaks and the area under the peaks associated with the gelatin aromatic residues (δ=7.2-7.3 ppm).

### Photopolymerization

Polymers were dissolved in Dulbecco's Modified Eagle Medium (DMEM) (8% w/v) at specified temperatures (PVAMA: 90° C., TPVA: 80° C., and GelMA: 60° C.) to obtain polymer solutions. Hydrogels were prepared by mixing TPVA, PVAMA, and GelMA at varied ratios, Table 1. Lithium phenyl-2,4,6-trimethylbenzoylphosphinate (LAP, 0.2% w/w) was added to the solution before thoroughly mixed by using a SpeedMixer (FlackTek Inc., 3500 RPM, 60 seconds). Then, samples were place in a UV box to start the crosslinking process (365 nm, 180 s), as seen in FIG. 1.

To acquire porous hydrogel (+pores), sodium bicarbonate (NaHCO3, 10 mg/ml) was dissolved in a 1 PVAMA: 4 TPVA: 2 GelMa polymer solution. Then, 950 μl of the solution was transferred into a 48 well plate, and 50 μl of 5% citric acid was pipetted into the bottom of the well. The solution was mixed thoroughly using a spatula, and then immediately placed into the UV box for curing as described above. For naming purpose, samples are defined with “PVA” followed by the ratio of TPVA:PVAMA employed during crosslinking. Samples without GelMA and pores are labeled as “−G/−P.” Samples with GelMA and without pores are labeled as “+G/−P.” Samples with GelMA and pores are labeled as “+G/+P” (Table 1). PVA14 was employed as the base for the addition of GelMA and pores, based on preliminary studies showing that it had the slowest degradation rate, minimizing the potential effects of degradation on study results.

TABLE 1
Hydrogel components in 1000 μl volume samples for the six formulations

| | | | | | | Citric |
|---|---|---|---|---|---|---|
| | | TPVA | PVAMA | GelMA | NaHCO₃ | Acid |
| Sample ID | TPVA:PVAMA:GelMA | (μl) | (μl) | (μl) | (mg) | (μl) |
| PVA11 | 1:1:0 | 500 | 500 | X | X | X |
| PVA12 | 1:2:0 | 333 | 666 | X | X | X |
| PVA13 | 1:3:0 | 250 | 750 | X | X | X |
| PVA14 | 1:4:0 | 200 | 800 | X | X | X |
| (−GelMA/−Pores) | | | | | | |
| PVA14 | 1:4:2 | 143 | 572 | 285 | X | X |
| (+GelMA/−Pores) | | | | | | |
| PVA14 | 1:4:2 | 136 | 544 | 270 | 10 | 50 |
| (+GelMA/+Pores) | | | | | | |

TPVA, PVAMA, and GelMA were all added at 80 mg/ml.
NaHCO₃ was added at 10 mg/ml.
Citric acid was added a 5 vol %.

### Hydrogel Characterization

Cylindrical samples (n=3, ˜10 mm diameter, ˜5 mm height) were completely dried under vacuum (50° C., 24 hr) after crosslinking. Samples were then weighed (Wi), placed in a 24 well plate with DI water, and left on a shaker table (60 rpm, 37° C.) for 24 hr. Samples were weighed again in the wet state (Ww). Samples were completely dried again before being weighed again (Wf). Swelling ratio and gel fraction were calculated using the equations:

$$Gel Fraction ( \% ) = \frac{{W}_{f}}{Wi} \times 100$$

$$Swelling ratio ( \% ) = \frac{Ww - Wi}{Wi} \times 100$$

The Young's modulus of all samples was acquired by performing compression tests (Model 100P Universal, Test Resources, Shakopee, MN). Hydrated cylindrical samples (n=3) fabricated in a 48 well plate mold were employed for the trials. Young's modulus was determined based on the slope of the linear elastic region in resulting stress-strain curves.

### Hydrogel Microstructures

The microstructures of hydrogel samples with and without pores were visualized using scanning electron microscopy (SEM, JCM 5000, Jeol NeoScope). The samples were frozen at −80° C. for 24 hr immediately after crosslinking. The frozen hydrogels were then lyophilized (FreeZone 2.5 Liter, Labconco Corporation, MO) for 48 hr to freeze-dry. Samples were cut into 6×6×1 mm pieces and coated with gold for 45 s using a high vacuum sputter coater (Denton). Microstructures were observed under 10 kV accelerating voltage at 35× magnification. ImageJ was utilized to measure relative pore size of hydrogel images.

### Hydrogel Degradation Profiles

The degradation profiles of hydrogels were assessed in PBS (‘real-time’ hydrolytic), 0.1M NaOH (accelerated hydrolytic), 3% H2O2 (‘real-time’ oxidative), and Dulbecco's Modified Eagle Medium (DMEM). After samples were formed into cylinders (˜10 mm diameter, ˜6 mm height), initial wet weights (Wi) were recorded before placing into media (n=3). All samples were kept in a shaker table at 37° C. Sample masses were measured every 3days (W_n) with fresh solutions provided every 2 days. The swollen mass remaining was determined at each time point using the equation:

$$Swollen Mass Remaining ( \% ) = \frac{{W}_{n}}{{W}_{i}} * 100$$

### Cytocompatibility

Initial testing was carried out using NIH/3T3 fibroblasts to ensure basic cytocompatibility of the synthesized materials. NIH/3T3 fibroblasts (passage 9) were cultured in DMEM supplemented with 10% fetal bovine serum (FBS) and 1% penicillin/streptomycin (Pen/Strep). Twenty-four hours before testing, cells were seeded into 24 well plates (5000 cells/well). Dried cylindrical samples (n=3, ˜5 mm diameter, ˜2 mm height) were sterilized under UV light for 30 mins. Then, samples were placed in Transwell® inserts and incubated with the pre-seeded cells for 24 hr. A LIVE/DEAD™ assay (Thermofisher) was employed to assess cell viability. Briefly, a working solution was prepared based on the manufacturer protocol (2 μM calcein AM, 4 μM ethidium homodimer-1). Cells were incubated with the working solution for 30 min at room temperature. Images of cells (green: alive, red: dead) were taken using an inverted microscope (Leica DMI6000B, Leica Microsystems Inc., Deerfield, IL). ImageJ was used to quantify the viability of cells. Cells only exposed to media were used as positive (cytocompatible) controls, while 70% ethanol was added to cells to provide a negative (cytotoxic) control.

### Cell Encapsulation

A375 epithelial cells (ATCC) were cultured as described above for the cell encapsulation experiment. Briefly, A375 cells (Passage 5, 3.3×10\hat{ }5 cells/ml) were suspended in three types of sample solutions before crosslinking (−GelMA/−pores, +GelMA/−pores, +GelMA/+pores). All solutions were mixed gently in a 48 well plate and then placed under UV light for 180 s. Cylindrical samples were obtained with dimensions of approximately 10 mm height and 8 mm diameter. A subset of samples was sliced to obtain cylinders at three heights (1 mm, 5 mm, 10 mm), and then hydrogels were placed in media with fresh media supplied every 3 days.

A375 epithelial cell viabilities was assessed at day 7 and day 14 using a live/dead assay. For 5 mm and 10 mm samples, hydrogels were divided into three sections (top, middle, bottom), while 1 mm samples were kept as-is. An inverted microscope (10×) was applied to take pictures at 5 random positions in each location.

SEM was used to further observe hydrogel structures after cell encapsulation. Samples were fixed using 2.5% glutaraldehyde at 4° C. for 48 hr and then washed twice using PBS. Samples were dehydrated by washing with 30%, 50%, 70%, 90%, 100% ethanol (30 mins/each step). Samples was left into vacuum oven at room temperature (24 hr) before sputter coating and imaging as described above.

In addition, the +GelMA/+pores sample was characterized using encapsulated mesenchymal stem cells (MSCs). Briefly, MSCs was encapsulated into the hydrogel at 50,000 cells/ml as described above. After 14 days of culture, samples were fixed with 4% paraformaldehyde. Cells were stained with Acti-Stain 555 Phalloidin (PHDH1, Cytoskeleton, Inc) to observe the filament structure of MSCs. Anti-CD90 (ab181469, Abcam) was employed to further confirm MSC characteristics. To observe nuclei, 4′,6-diamidino-2-phenylindole dihydrochloride (DAPI, Invitrogen) was also applied. Images were taken using a confocal microscope (Zeiss LSM980, ZEISS Microscopy).

3T3/NIH green fluorescent protein (GFP) fibroblasts were encapsulated into three types of samples (−GelMA/−pores, +GelMA/−pores, +GelMA/+pores) to characterize proliferation capacity over time. The encapsulation process was carried out described above. Samples was observed at days 1, 4, 7, and 14 using Leica Thunder Microscope. Imaging was carried out at 10× magnification, exposure 40, with z-stack of 501.05 μm. ImageJ was employed to calculate cell number and area over time.

### Cell Proliferation

NIH/3T3 GFP fibroblasts were seeded in 24 well-plates at 5,000 cells/cm\hat{ }2 and cultured for ˜3 days in DMEM with 10% FBS and 1% Pen/Strep. When cells reached 100% confluency, cell media was removed, and a ‘scratch’ was created in the middle of the cell monolayer using a 10 μL pipette tip. Cell media was used to wash off debris. Sterilized samples (PVA14 −GelMA/−Pores/−MSC, PVA14 +GelMA/−Pores/−MSC, PVA14 +GelMA/−Pores/+MSC, PVA14 +GelMA/+Pores/+MSC) were placed in Transwell® inserts above the damaged cells, and media was supplied to make sure adequate indirect contact between samples and cells. Media was employed as control, Platelet Derived Growth Factor-BB was used as positive control, and DMSO (2% in media) was utilized as negative control. Images were taken at 0 hr and 24 hr using an inverted microscope (5×). ImageJ was used to measure the percentage of cell coverage of the wound area at each time point.

NIH/3T3 GFP fibroblasts were employed in the scratch wound healing assay to improve imaging efficiency and enable nondestructive imaging of samples at multiple time points. Cells were seeded in 24 well-plates at 5000 cells/cm2 and cultured for ˜3 days in DMEM with 10% FBS and 1% Pen/Strep. When cells reached 100% confluency, cell media were removed, and a “scratch” was created in the middle of the cell monolayer using a 10 μL pipette tip. Cell media were used to wash off debris. Sterilized samples (All PVA14: −G/−P, +G/−P, without [−M] and with [+M] encapsulated MSCs, and +G/+P/+M [with encapsulated MSCs]) were placed in Transwell inserts above the damaged cells, and media were supplied to make sure adequate indirect contact between samples and cells. Media were employed as control, platelet derived growth factor (PDGF) was used as positive control, and 2 vol % DMSO in media was utilized as negative control. Images were taken at 0 and 24 h using an inverted microscope (5×). ImageJ was used to measure the percentage of cell coverage of the wound area at each time point.

### Ex Vivo Wound Healing Assay

Porcine skin has a morphology similar to that of human skin, and ex vivo models include the full skin structure. The ex vivo porcine skin model protocol was adapted from a previous study. The aim is to keep the porcine skin cells alive during the study to enable characterization of their proliferation after wound treatment. Tissue for the ex vivo wound healing model was obtained from porcine ears using a tissue sharing protocol with SUNY Upstate Medical University, protocol number 332, 478. After animals were sacrificed as part of another study, the ears were immediately cut off and stored at −80° C. After thawing, the hair was removed using a razor blade. The ear was thoroughly washed in water, dipped into 70% ethanol, and then rinsed with sterile 0.9% NaCl. Wounds were made using 3 mm biopsy punches to remove the epidermis and top dermis (estimated depth of 1.5 mm). The area around the wounds were then cut into 1×1 cm pieces using a scalpel and transferred into 12-well plates. The skin was cultured in DMEM, 10% FBS, 1% Pen/Strep. Wounds were treated with five types of samples including media only, PVA14 −G/−P, +G/−P/−M, +G/−P/+M, and +G/+P/+M. Samples were collected for analysis in triplicate at days 0 (for control), 1, and 3. After collecting, samples were fixed in 4% formaldehyde in PBS, pH 7.4 at room temperature for 15 min then stored at 4° C. overnight before embedding in paraffin. Samples were then dehydrated before sectioning (10 μm). Parafilm was removed using a tissue clearing agent, and then the tissue was hydrated. Resulting slides were blocked with blocking buffer (10% FBS, 1% BSA, 1× PBS). Ki-67 Recombinant Rabbit Monoclonal Antibody (MA5-14520, Thermo Fisher) was applied overnight at 4° C. as a primary antibody to identify proliferative cells. Goat anti-Rabbit IgG (H +L) Cross-Adsorbed Secondary Antibody, Alexa Fluor 488 (A-11008, Thermo Fisher) was applied as a secondary antibody for 1 h at room temperature. DAPI was applied for 5 min to stain cell nuclei. The coverslip was then mounted with Entellan for storage and imaging. An inverted microscope was employed to take images, and the number of proliferative cells over the epidermis area (estimated to be the top 50 μm of the wound surface) was calculated using ImageJ.

All experiments were performed triplicate (n=3). One-way ANOVA, followed by student's T-test between groups was acquired. Significance was determined at p≤0.05, ≤0.01, or ≤0.001. Mean±standard deviation is displayed for results.

### Hydrogel Components

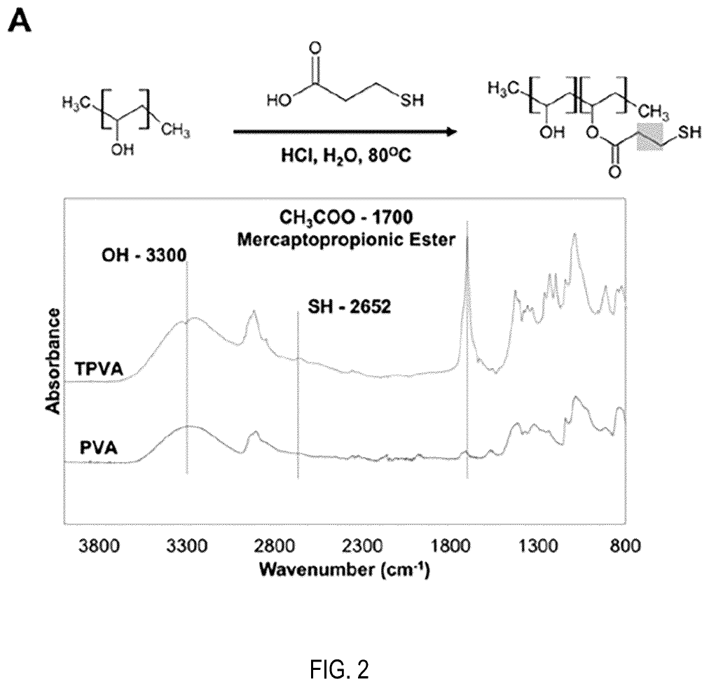

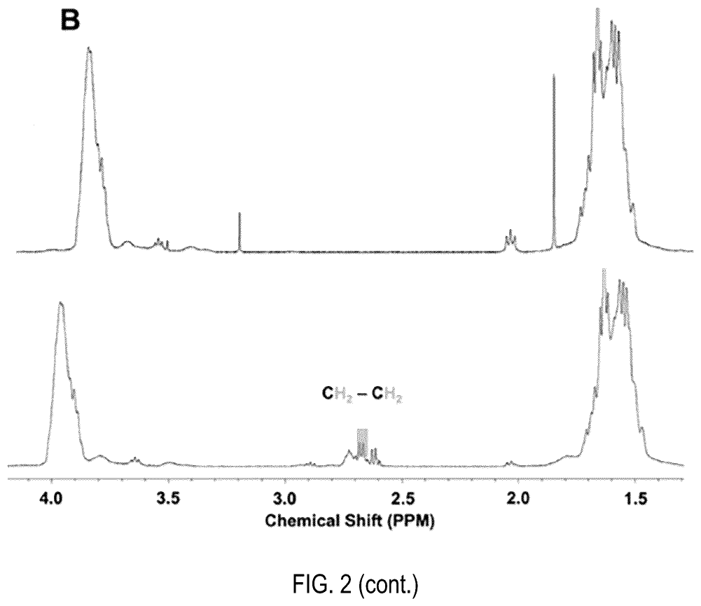

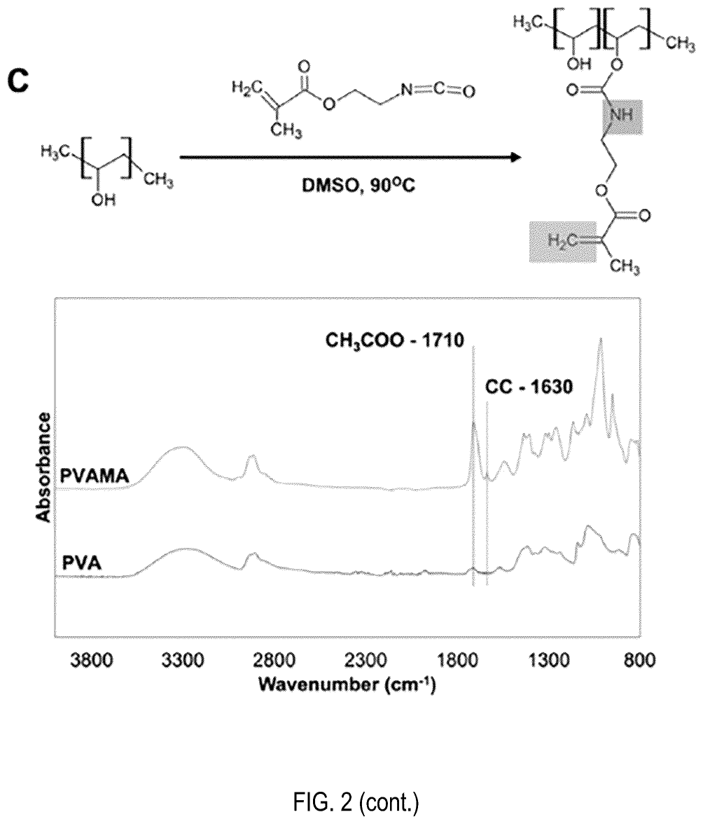

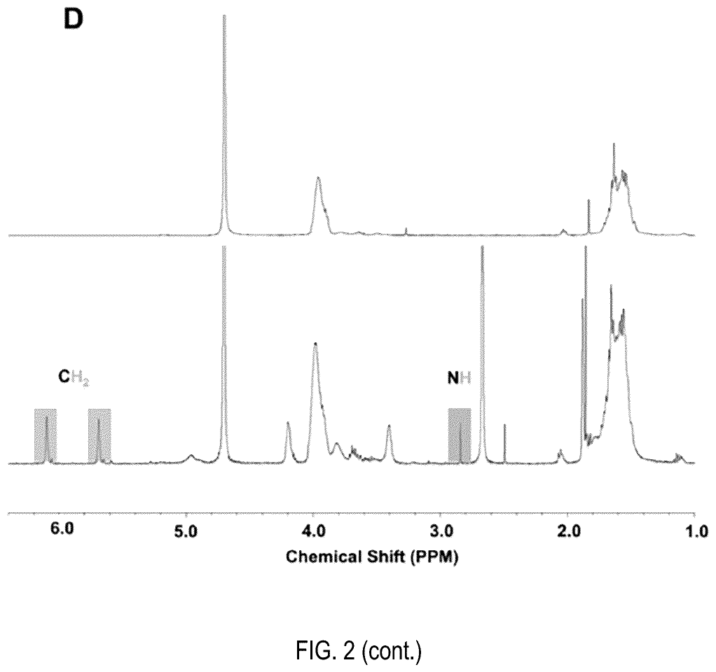

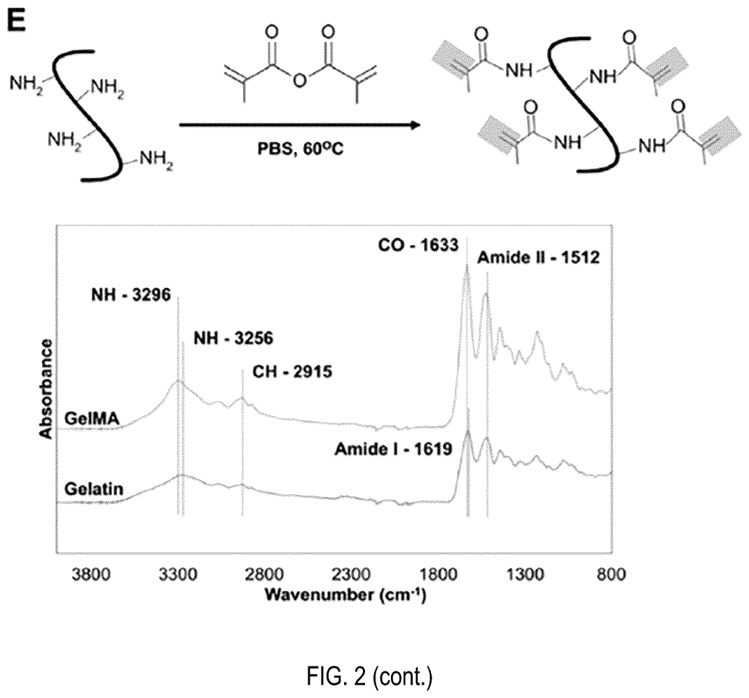

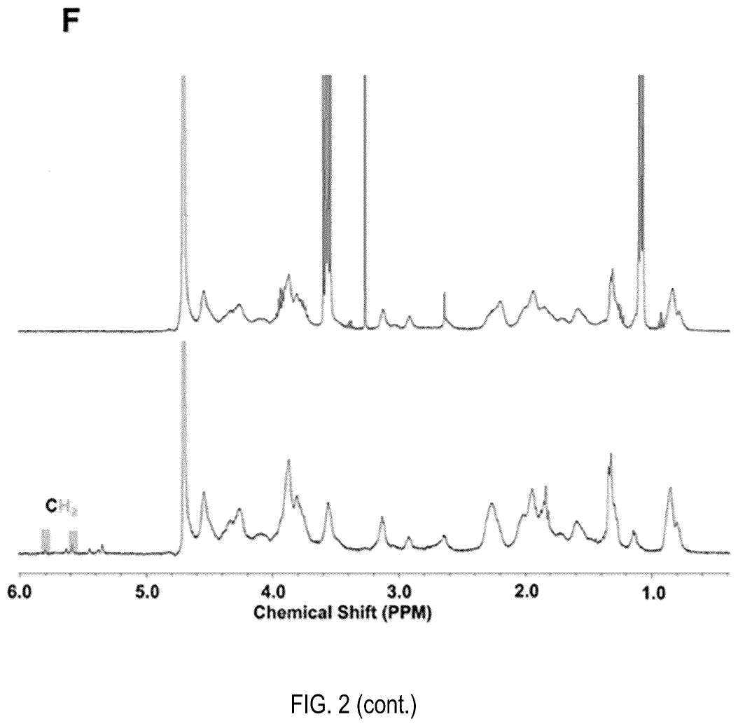

FTIR and NMR were employed to confirm synthesis of hydrogel components. The FTIR spectra of TPVA in FIG. 2A shows the S—H peak at ˜2652 cm−1 and mercaptopropionic ester C═O peak at ˜1700 cm−1 in addition to key PVA peaks (OH at ˜3000-3600 cm−1) and therefore demonstrates successful synthesis of TPVA from PVA. FIG. 2B displays the NMR analysis of TPVA. Successful synthesis was further confirmed by the peak of the 3-mercaptopropionic ester proton at 2.7 ppm. The degree of substitution (DS) of thiol groups on TPVA was measured as approximately 3%.

The structure of PVAMA was confirmed based on the present of C═O of the ester at ˜1706 cm−1 and C-C double bond stretching at ˜1630 cm−1 (FIG. 2C). In the NMR spectra, PVAMA displayed vinyl group proton peaks at 5.6 and 6.2 ppm, along with an amine peak at 2.8 ppm, FIG. 2D. The DS of methacrylate groups on PVAMA was calculated as roughly 3%.

The FTIR spectra of synthesized GelMA, FIG. 2E, shows C-O stretching at ˜1628 cm−1, corresponding with amide I in gelatin structure at 1619 cm−1, which correspond with stretching vibration of N H and O H functional groups. The C H stretching at ˜2915 cm−1 was observed for both structures. The shift of N H bonds from ˜3256 to ˜3296 cm−1 confirmed the successful synthesis of GelMA. In addition, the C O bond peak in GelMA at ˜1633 cm−1 has a higher intensity compared with the amide peak of gelatin at ˜1619 cm−1, which has been previously utilized as confirmation of GelMA synthesis. NMR analysis shows peaks associated with the methacrylate double bond protons at 8=5.6 and 5.8 ppm, indicating successful synthesis of GelMA with DS at approximately 20% as seen in FIG. 2F

### Hydrogel Fabrication

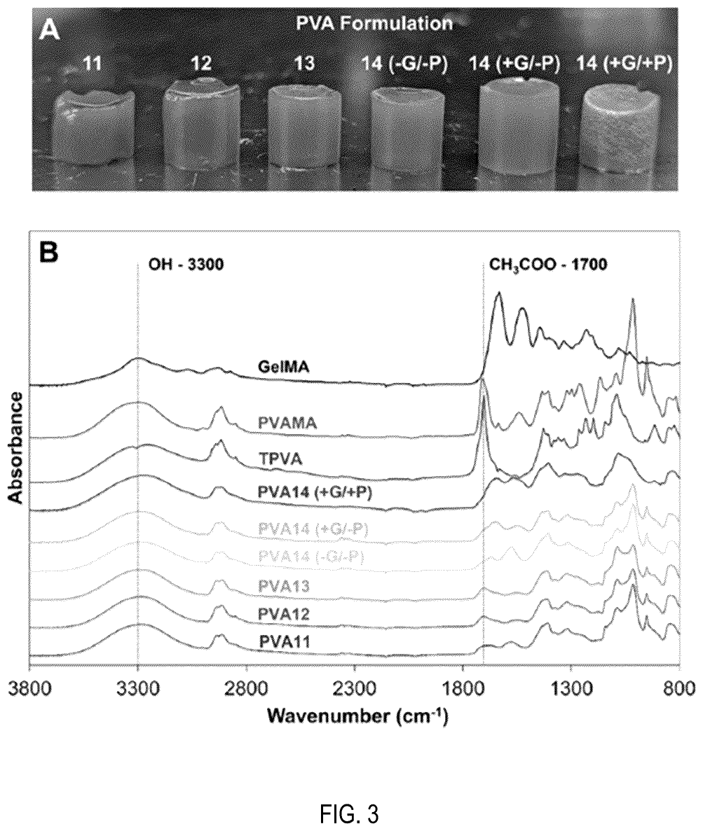

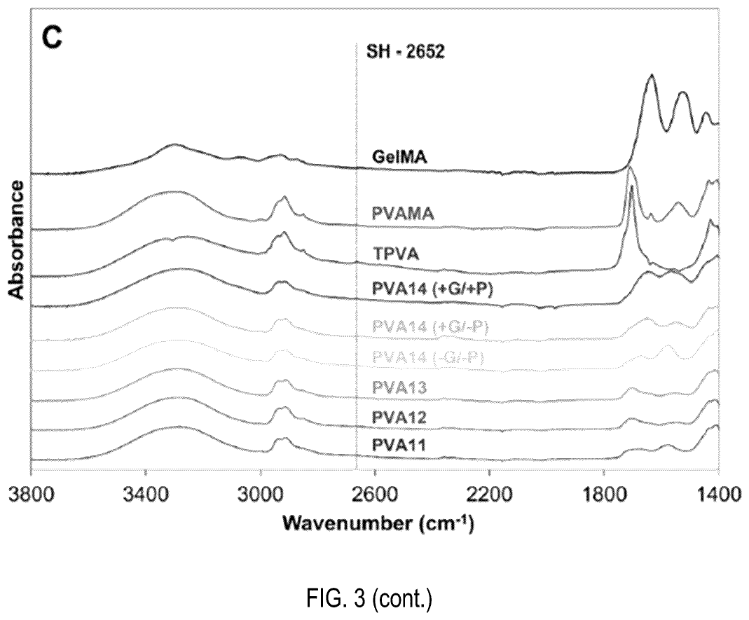

In the presence of UV light, the LAP photoinitiator generates radicals to initiate the thiol-ene coupling reaction, in which a thiol will react with an ene. After all of the thiols are consumed, the system will likely continue with the reaction between remaining double bonds in the methacrylate functional groups to form a crosslinked hydrogel. After crosslinking with UV light for 3 min, solid hydrogels were obtained (FIG. 3A). Hydrogels maintain the OH peak at ˜3300 cm−1 from the PVA backbone and the C O of the ester peak at ˜1700 cm−1 from the thiol methacrylate side groups (FIG. 3B). The C O of the ester peak in PVA14 (+G/−P) and PVA14 (+G/+P) was shifted compared with that of the other hydrogels. This change may be due to the influence of GelMA in these samples. To better analyze changes in the thiol groups after crosslinking, the FTIR spectra of hydrogels was more closely analyzed between 1400 and 3800 cm−1 in comparison with TPVA, PVAMA, and GelMA, as shown in FIG. 3C. The disappearance of the S H bond peak at ˜2652 cm−1 in all crosslinked hydrogels indicates successful complete thiol-ene reaction within this system.

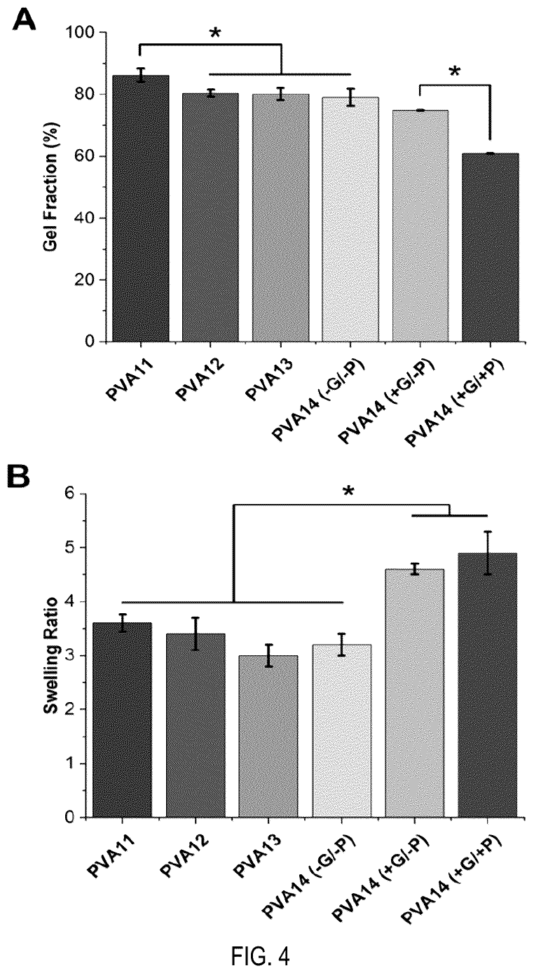

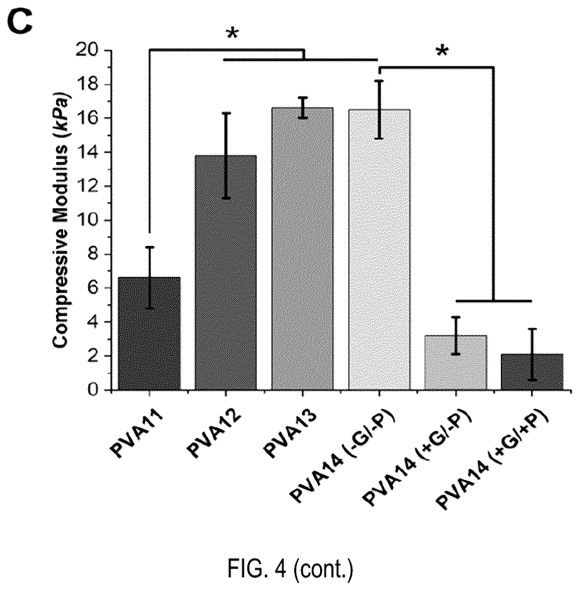

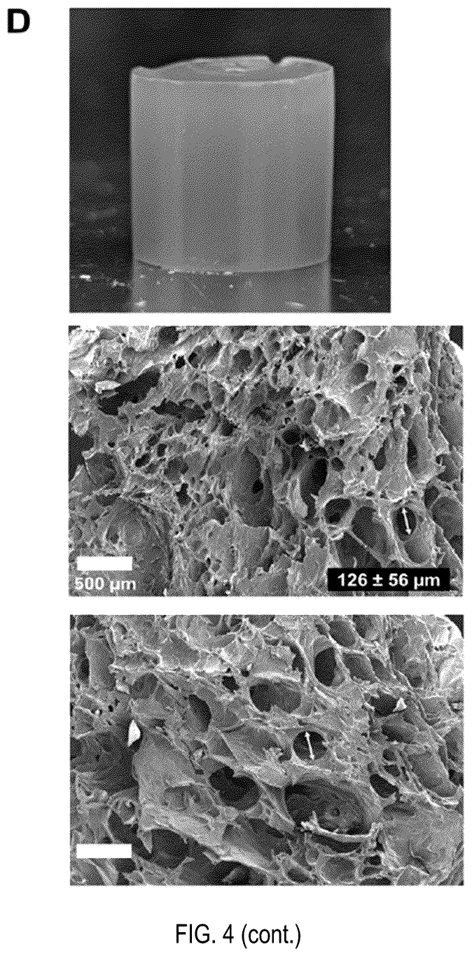

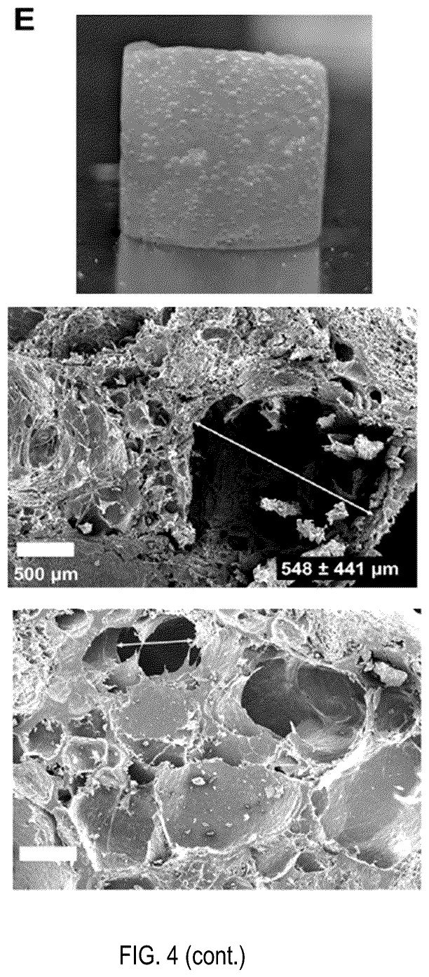

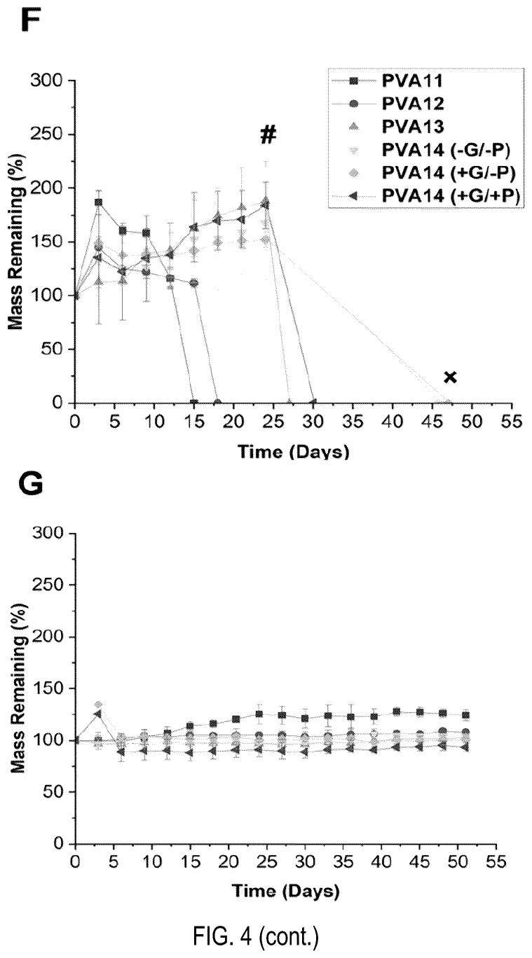

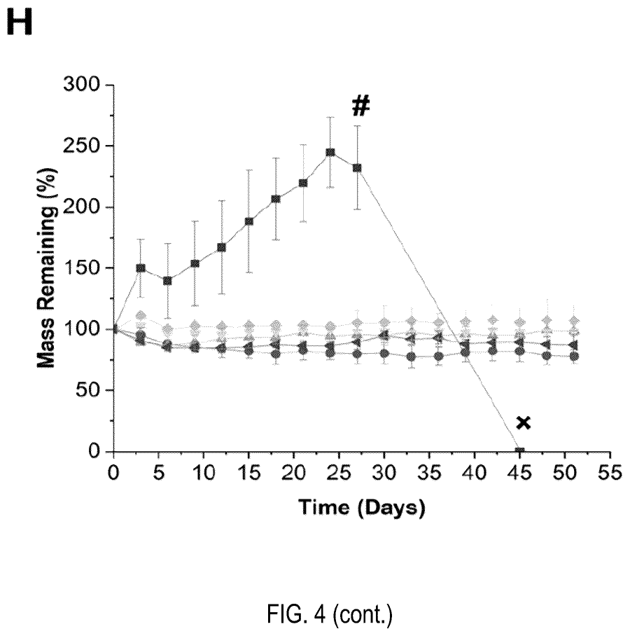

PVA14 (+G/+P) had significantly lower gel fraction percentage compared with the other formulations, which may be attributed to solution heterogeneity when combining the different components in the system (FIG. 4A). PVA11 had the highest gel fraction, indicating the high efficiency of the thiol-ene reaction. All other samples had gel fractions between 70% and 90%. In swelling analysis, samples with GelMA had significantly higher swelling ratio (FIG. 4B), which has been seen in prior literature and is attributed to the high number of hydrophilic/hydrogen bonding sites in GelMA. Altering TPVA:PVAMA content did not have any significant effects on swelling.

Within these synthesized hydrogels, PVA14 (+GelMA/−Pores) and PVA14 (+GelMA/+Pores) samples have the lowest compressive modulus at 2.1±1.5 and 3.2±1.1 kPa, respectively, as seen in FIG. 4C. This data correlate with the higher swelling ratios of these samples. Interestingly, the addition of pores did not affect modulus of these gels, despite its corresponding increase in free space in the hydrogels. PVA13 and PVA14 (−GelMA/−Pores) have highest modulus at 16.6±0.6 and 16.5±1.7 kPa, respectively. In general, as PVAMA content increased in hydrogels without GelMA, compressive modulus increased.

Thiol-ene reactions undergo ideal step-growth polymerization, which includes chain transfer and radical propagation. Thioether bonds will be formed until the termination stage is reached. Thus, it was expected that hydrogels with higher relative amounts of methacrylate groups (vs. thiol groups) will undergo a second potential reaction in which the remaining methacrylate double bonds react with each other. These secondary reactions will increase the crosslink density of the hydrogel to generate a higher modulus.

Previous studies of the mechanical properties of thiol-ene hydrogels demonstrate that mesh size could also impact the bulk modulus. The hydrogels with GelMA have lower modulus and higher swelling ratio, indicating that they may have a larger mesh size that could advance nutrient transfer in the system. Some have suggested that high DM could produce poor mechanical properties due to inhibition of the formation of a strong physical network. Also, the modification process in gelatin reduces the number of helices and produces weaker hydrogels due to obstructed helix formation.

Overall, the compressive testing experiments confirmed that tunable stiffnesses of TPVAGelMA hydrogels could be achieved by controlling the ratio of TPVA:PVAMA: GelMA without altering other variables. Notably, swelling and modulus are typically inversely related in hydrogel systems, where increased modulus is only observed with simultaneous decreases in swelling. Here, swelling was held constant while modulus was tuned over a wide range. Modulus is an important factor in hydrogels for cell encapsulation. Some researchers have demonstrated that hydrogel modulus affects the proliferation and differentiation of encapsulated stem cells. As hydrogel modulus has a profound impact on the behavior of encapsulated cells, the present invention system could provide a unique advantage for various cell encapsulation applications. Future work will investigate the relationship between bulk and cell-scale hydrogel modulus and how these variables affect cell interactions.

Microstructure also plays a role in encapsulated cell behavior; thus, hydrogels were imaged via SEM to visualize their internal structures. With solid hydrogels synthesized without employing the foaming process (i.e., PVA14 (+G/−P)), resulting microstructures are uniform with small pores (126±56 μm) (FIG. 4D). Upon adding pores to the hydrogel using gas blowing, with PVA14 (+G/+P), pore sizes are less uniform, and large pores are evident (548±441 μm) around the regular hydrogel microstructure (FIG. 4E). Hydrogel pore size plays an essential role in cell interactions and vascularization processes. Some researchers showed that freeze-dried scaffolds with pore size of ˜280 μm are suitable for homogenous cell seeding. Others utilized gelatin beads to control hydrogel porosity to produce pore sizes between 150 and 300 μm, which increased solution permeability by two-fold. Some have found that 200-250 μm diameter pores were ideal for endothelial cell migration and vascularization of thick hydrogel constructs. The current invention unveiled that PVA14 (+G/+P) employs a rapid, simple method to fabricate hydrogels with a desirable pore size, which could advance permeability capacity in cell encapsulation applications.

### Degradation Profiles

All hydrogel samples were fully degraded via oxidation in H₂O₂ after 47 days, as seen in FIG. 4F. As hydrogels degrade, their crosslink density decreases, which results in increased swollen masses over time when submerged into oxidative solution. Oxidative degradation occurred more slowly with increased ratios of PVAMA relative to TPVA. TPVA11 and TPVA12 underwent the fastest degradation, with full dissolution occurring at days 15 and 18, respectively. The hydrogels underwent oxidative degradation due to the thioether (sulfide) CH2 S CH2 structure. The presence of H2O2 converts these groups into sulfoxides and sulfones, which are more hydrophilic. Increased TPVA will increase the portion of sulfide bonds in the system, which allows faster oxidative degradation.

PVA14 (−G/−P) and PVA14 (+G/−P) degraded the slowest (46-47 days) due to decreased sulfide bonds. The addition of pores to PVA14 (+G/+P) sped up degradation, with full dissolution at 30 days. Wu et al. showed that porosity and pore size influence the degradation rate due to the change in wall thickness and surface area. Scaffolds with pore sizes between 180 and 450 μm degraded faster than scaffolds with smaller pore sizes (50-180 μm). Similarly, Odelius et al. indicated that higher pore sizes produce higher mass loss among porous scaffolds. Thus, introduction of pores into these hydrogels provides a mechanism for tuning degradation rates independently of chemistry, which is attributed to increased solution transfer in and out of the gels during degradation.

In this system, sulfide oxidation facilitates adjacent ester hydrolysis. Thus, characterization of degradation in 3% H2O2 will induce both oxidation and hydrolysis, which is representative of what the materials would experience in the wound environment. To characterize hydrolysis independently of oxidation, PBS and 0.1 M NaOH were employed. In general, these hydrogels demonstrated high hydrolytic stability without the presence of ROS, under “real time” and accelerated conditions (FIG. 4G,H). In “real-time” conditions, at Day 51, the PVA11 hydrogel mas remaining was significantly higher than that of other samples, which could be due to hydrolytic cleavage of ester bonds to increase swelling (FIG. 4G). To better understand hydrolytic degradation, an accelerated study was conducted in 0.1 M NaOH. Again, minimal mass changes were observed over 51 days of degradation in all samples except for PVA11 (FIG. 4H). The increased ratio of TPVA in PVA11 resulted in immediate increases in swollen mass as the crosslink density decreased during degradation to increase swelling capabilities. The PVA11 hydrogels were fully degraded after 45 days in 0.1 M NaOH. For other samples, the reaction between remaining methacrylate double bonds could increase the crosslink density and hydrolytic stability to influence the degradation time. Samples with GelMA show similar trends in prior literature, due to its hydrolytic stability. Controlling the ratio of TPVA in the hydrogels of the present invention can be used to alter the degradation rate without changing other variables to yield a system for a range of desired applications.

### Cytocompatibility and Cell Encapsulation

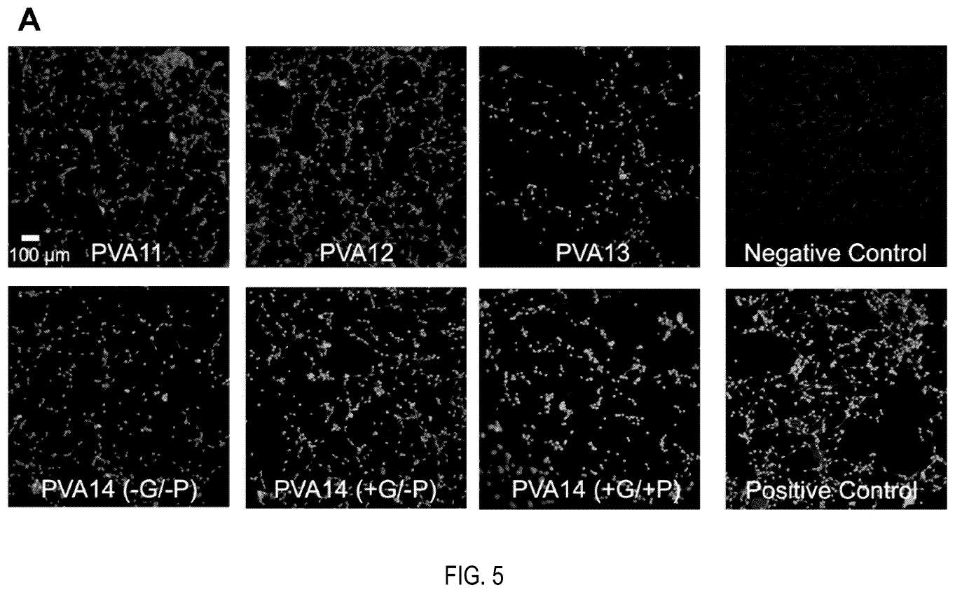

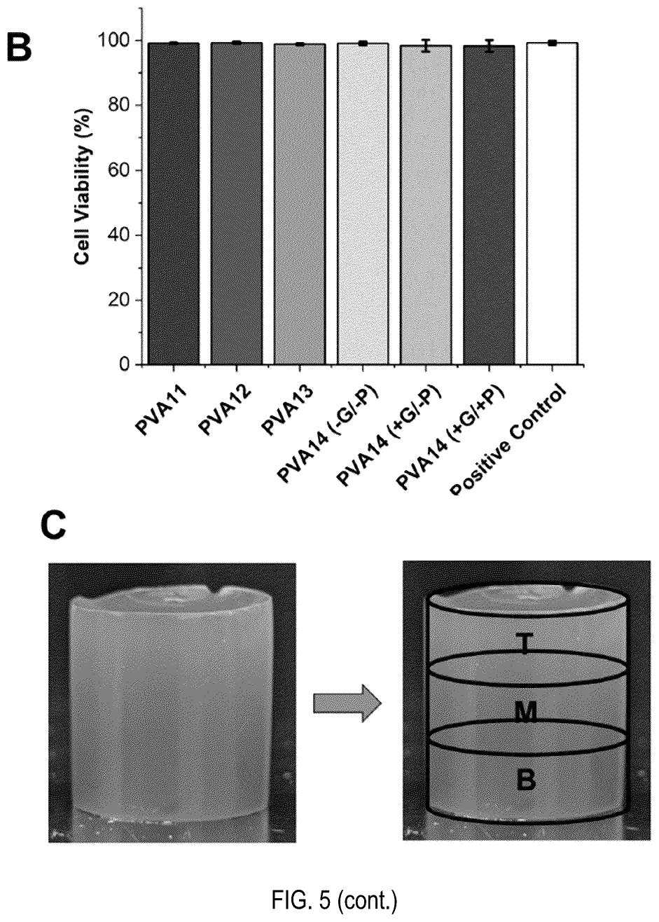

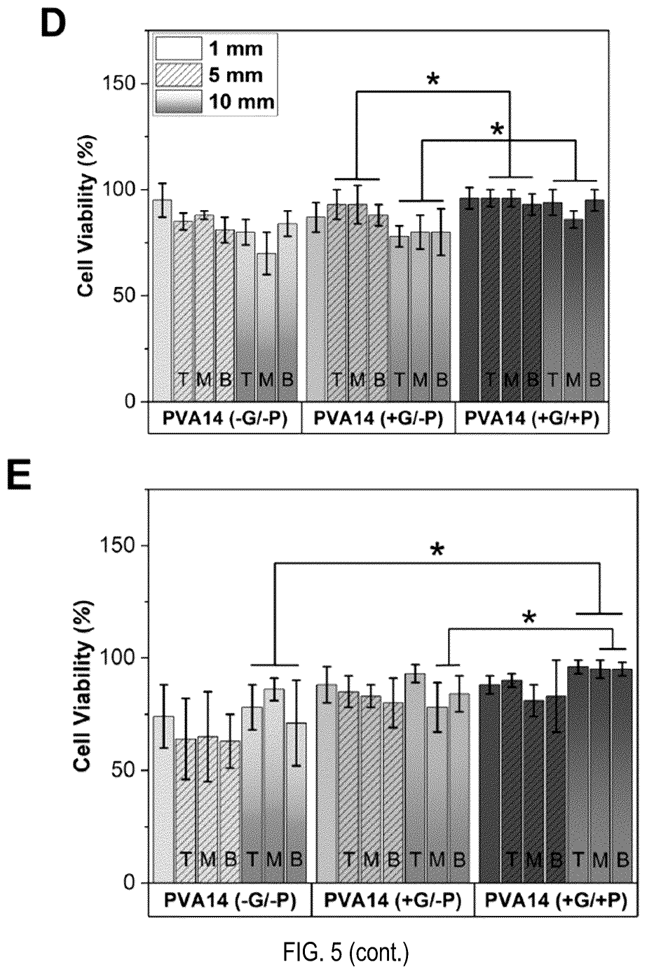

All samples showed high cell viability (>95%) over 24 hours of testing, FIG. 5A, B. Upon confirming cytocompatibility, A375 epithelial cells were encapsulated within the PVA14 hydrogels during fabrication, and viability was assessed over 2 weeks based on hydrogel thickness. A375 cells were selected here based on their high proliferative capacity to assist in establishing a proof-of-concept for cell encapsulation within the hydrogel system of the present invention. PVA14 hydrogels were employed for encapsulation due to their minimal mass changes in hydrolytic degradation media to enable investigation of long-term cell viability independently of degradation processes. Cell viability was assessed in three different planes Top (T), Middle (M), and Bottom (B) for all samples (FIG. 5C).

At Day 7, all samples have relatively high cell viability throughout their full thicknesses, ranging from 70-95%. In the 1 mm samples, PVA14 (+G/+P) had significantly higher cell viability than PVA14 (+G/−P). Interestingly, there was no significant difference in cell viability among the PVA14 (+G/+P) and PVA14 (−G/−P) (FIG. 5D). In the 5 mm samples, PVA14 (+G/+P) had significantly higher cell viability compared with PVA14 (−G/−P) in all positions throughout the sample thickness. PVA14 (+G/−P) only had higher cell viability compared with PVA14 (−G/−P) in the top (T) position. In the 10 mm samples, PVA14 (+G/+P) had significantly higher cell viability than PVA14 (−G/−P). When comparing positions within the samples, there was no significant difference in cell viability between PVA14 (+G/−P) and PVA14 (−G/−P).

After 14 days, larger differences in encapsulated A375 cell viability begin to emerge (FIG. 5E). In 5 mm hydrogels, PVA14 (+G/+P) had better cell viability than PVA14 (−G/−P) in both the top (T) and bottom (B) positions. PVA14 (+G/−P) only had higher cell viability than PVA14 (−G/−P) in the top (T) position. In the thicker hydrogels (10 mm), PVA14 (+G/+P) had significantly higher viability than PVA14 (−G/−P) in all positions. GelMA samples without pores only had significantly higher cell viability than PVA14 (−G/−P) in the top (T) position. In addition, in GelMA samples, hydrogel with pores provided significantly higher cell viability than hydrogels without pores in the middle (M) and bottom (B) positions. With long-term encapsulation, the thickness of hydrogel scaffold influences the cell viability. Increasing the thickness of hydrogels typically decreases the permeability of scaffolds. Hydrogel permeability permits nutrient transfer to secure cell development while allowing waste removal. These activities are based on diffusion of media around the hydrogel. Porous structures in the hydrogel system yield shorter diffusion distances that could improve nutrient and oxygen transfer to promote the observed improvements in cell viability. The foaming process and its components are highly important for cell survival in super porous hydrogels. PVA14 (+G/+P) outperformed PVA14 (−P) in maintaining high cell viability, demonstrating that the combination of sodium bicarbonate and citric acid in the system of the present invention during foaming is robust and safe for cells while generating adequate porous structures to maintain high cell survival rates. differences in encapsulated cell viability begin to emerge, FIG. 4c. Namely, thicker (10 mm) PVA14 (+GelMA/+Pores) samples have significantly higher viability than the 10 mm thick PVA14 (−GelMA/−Pores). This result suggests that hydrogels with both GelMa and porous structures support longer-term cell viability in larger scaffolds. Encapsulated Cell Proliferation and Morphology

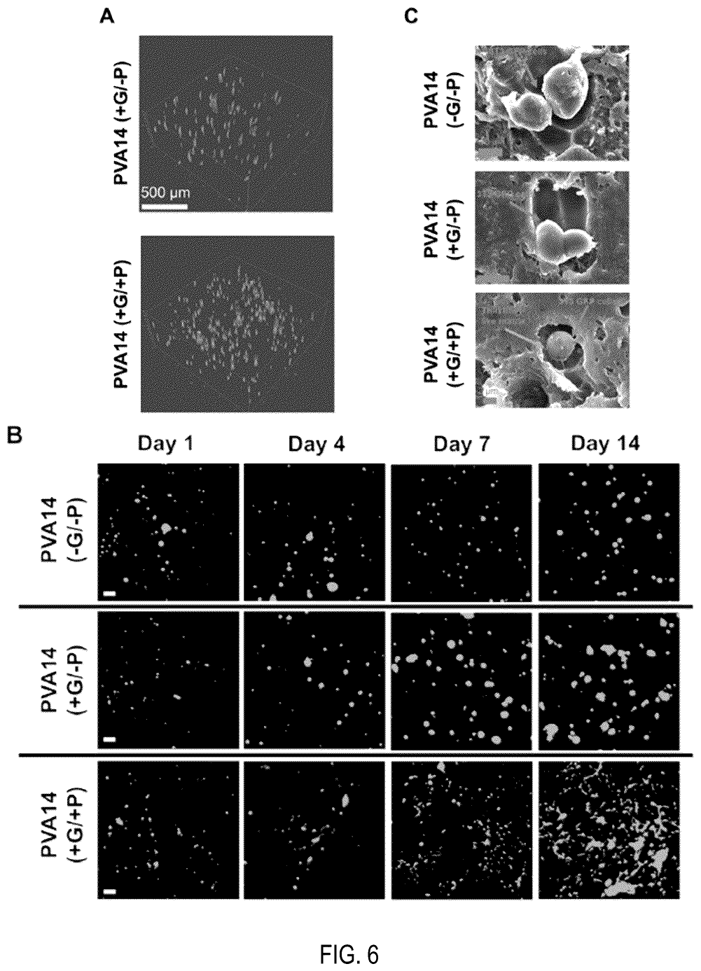

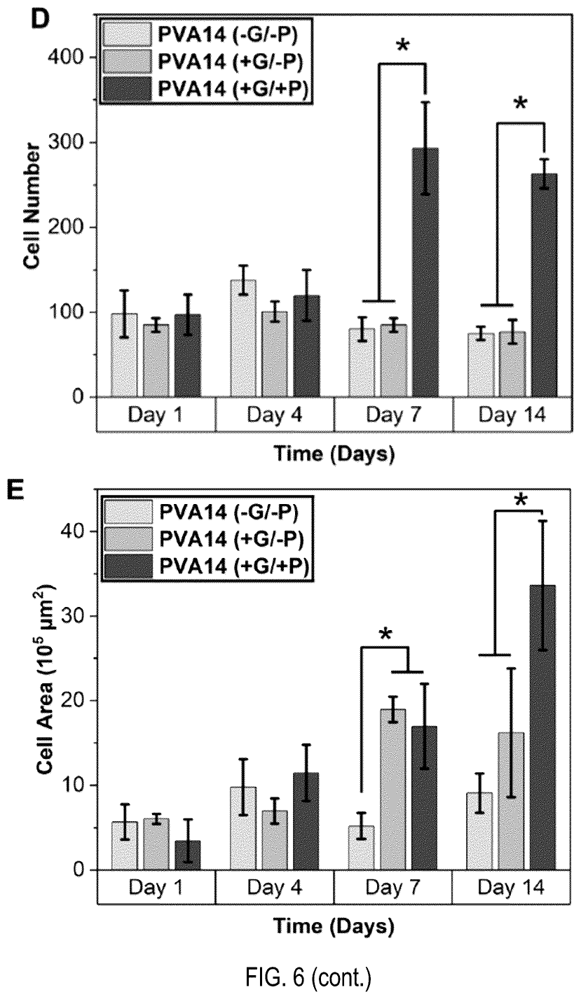

Upon confirmation of high long-term encapsulated cell viability, PVA14 (+GelMA/+Pores), PVA14 (−GelMA/−Pores), and PVA14 (+GelMA/−Pores) were employed to encapsulate 3T3/GFP fibroblasts to analyze proliferation over 14 days. The fluorescently labeled cells enable facile tracking of cell density over time in a single sample. FIG. 6A shows equal distribution of cells in 3-dimensions in the system of the present invention, overcoming the existing challenge in which inhomogeneous distribution of encapsulated cells causes different cell coverage within scaffolds. PVA14 (+G/+P) hydrogels had significantly higher cell numbers as compared with both PVA14 (−G/−P) and PVA14 (+G/−P) at days 7 and 14 (FIG. 6B,D). Additionally, on Day 14, cells within PVA14 (+G/+P) gels had significantly higher spreading than those in PVA14 (−G/−P) and PVA14 (+G/−P) (FIG. 6B,E). This data indicate that the addition of both GelMA and pores to PVA14 support cell proliferation during long-term encapsulation.

SEM images show how 3T3/GFP cells arranged themselves into the hydrogel microstructures. The cells encapsulated in PVA14 (+G/+P) attached to the material via a thin fiber-like matrix, which was not observed in other samples (FIG. 6C). GelMA promotes cell attachment due to its natural bioactive motifs, especially arginine glycine aspartic acid (RGD). Researchers have demonstrated that porous hydrogels support improved cell proliferation, which could be attributed to increased surface area after pore introduction. Taken together, these results show that hydrogels with both GelMA and porous structures provide uniform cell distribution while supporting longer term cell viability in larger scaffolds. This capability is especially important when considering the rapid fabrication process used here, wherein porous, cell-laden hydrogels are synthesized within <10 min without the need for specialized equipment (e.g., bioreactors) to ensure even cell distribution.

### 2D Scratch Closure

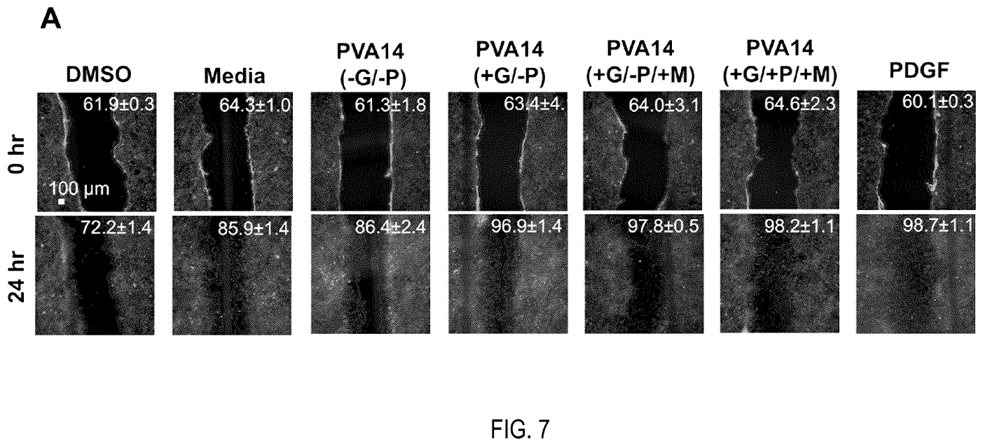

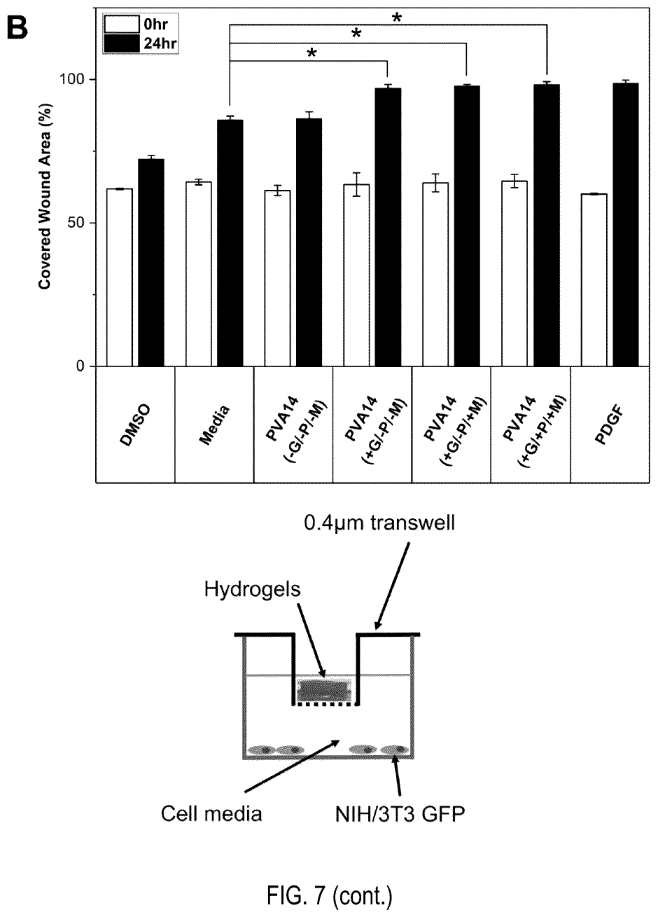

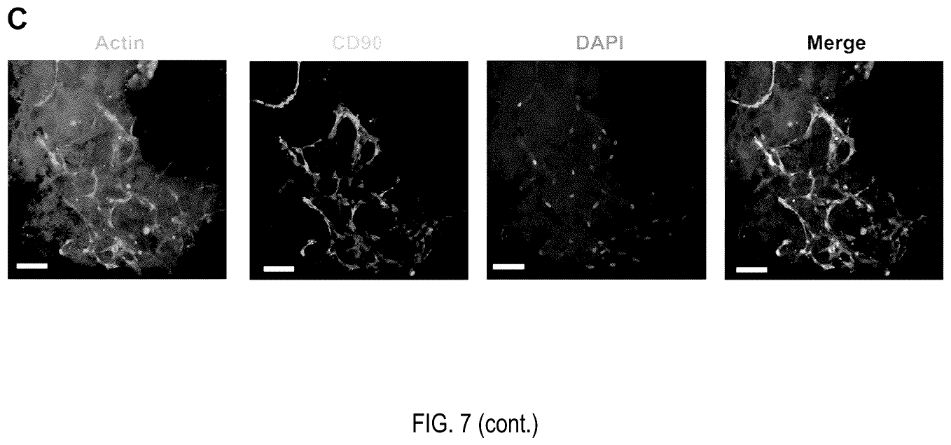

The 2D scratch assay is a simple system that mimics the repair process during re-epithelialization in wound healing. A library of PVA14 hydrogels was applied to 2D scratch wounds in vitro to analyze how they affected cell closure of the wounds (FIG. 7A). The PVA14 control did not impact scratch closure, with similar closure rates to the media-only control. The addition of GelMA with and without pores and/or encapsulated MSCs significantly enhanced wound closure to statistically similar areas to that of the positive control treated with PDGF (FIG. 7B). These results show that bioactive hydrogels with encapsulated MSCs could promote cell migration, which is supported by prior literature. The mechanism of MSCs promoting wound healing has been attributed to their trans-differentiation into epithelial and dermal cells, and/or their paracrine effect in secreting soluble growth factors. Overall, the PVA14 hydrogels do not negatively impact wound closure. By incorporating bioactive factors (GelMA and/or MSCs), the hydrogels can improve cell closure of 2D in vitro wounds.

As a proof-of-concept to further understand the potential utility of the PVA14 (+G/+P) hydrogels, MSCs were encapsulated for 14 days and stained for mesenchymal markers, (FIG. 7C). Namely, MSCs are characterized by the expression of F-actin and CD90. The stains show that MSCs maintain these markers at 14 days, indicating that MSCs can preserve their structure and characteristic MSC markers during longer term encapsulation timeframes. This study further implies that the impact of MSCs on cell migration in the in vitro scratch assay was most likely due to their soluble growth factor release. Cell interactions with ECM and/or polymer substances could induce signaling cascades that initiate cell differentiation. In general, it is crucial to control the cell fate during cell/material interactions. Thus, the preserved MSC markers after encapsulation indicates that the components of PVA14 (+G/+P) will not likely trigger differentiation of MSCs, which could enable their use in regenerative medicine applications in which differentiation factors are introduced in a controlled manner.

### Ex Vivo Porcine Skin Healing

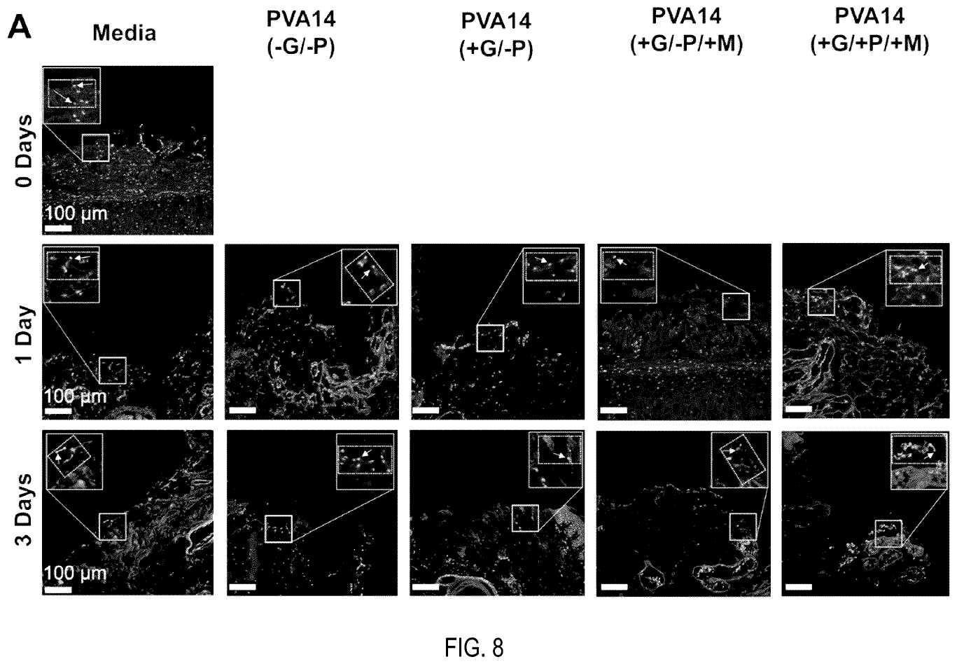

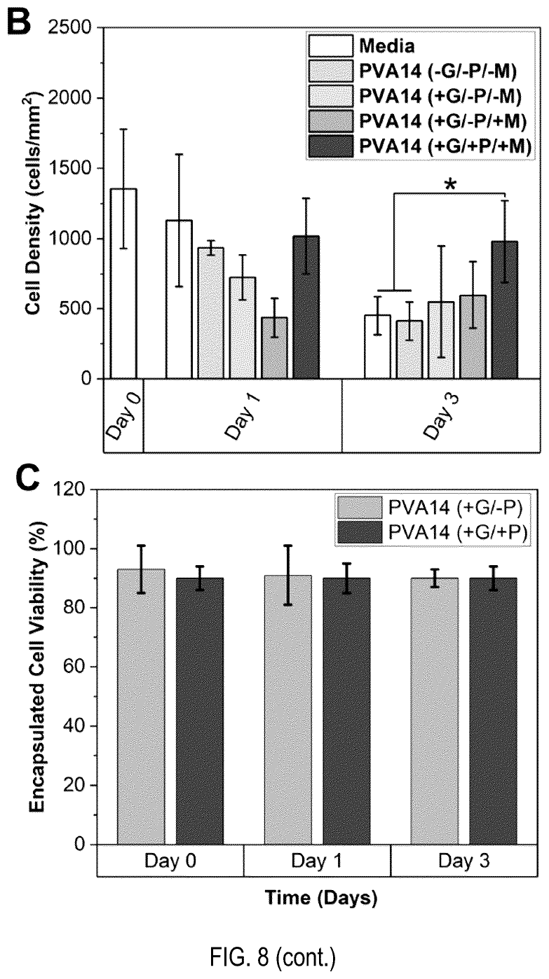

To better understand the effects of these hydrogels on wound healing, an ex vivo porcine skin wound model was employed (FIG. 8A). At Day 0, the average cell number of control samples was measured for reference (FIG. 8B). Overall, the number of active cells was decreased at Day 1 and Day 3 relative to that in the skin samples directly after wounding. This circumstance could be attributed to the nature of the ex vivo porcine skin, which is not connected to a blood supply. At Day 1, the number of active cells was similar between most of treatments. Interestingly, wounds treated with PVA14 (+G/−P/+M) have lowest cell numbers. At Day 3, samples treated with PVA14 (+G/+P/+M) had a significantly higher active cell number than samples treated with hydrogels without MSCs, indicating that MSCs encapsulated in the hydrogel of the present invention could help promote cell migration. As explained in scratch assay results, this performance is attributed to soluble factor secretion to support local cells. In addition, the porous structure could improve the diffusion of products from MSCs to the injured area.

Importantly, over the three-day study, encapsulated MSC viability was maintained at high levels in the stimulated injury environment (FIG. 8C), illustrating that the hydrogel system of the present invention is suitable for cell encapsulation and delivery to wound sites. Combined with the scratch assay data, these results indicated that these porous hydrogels with GelMA and MSCs could enhance wound healing processes by allowing rapid transfer of soluble factors from MSCs to the local damaged area. The aim of this work is to develop tunable, porous scaffolds to support MSC encapsulation, with a long-term goal of applying these materials to chronic wound dressing. The scratch assay and ex vivo porcine skin wound model suggest that this hydrogel system could promote healing in the short term. Thus, these results will serve as the foundation for future studies, where in vivo wound models will be employed to confirm these effects.

### Conclusion

The present invention thus provides a rapid method to fabricate hydrogel foams composed of PVA modified with methacrylate/thiol functional groups and GelMA, which is suitable for cell encapsulation. The porous structures improved solution transfer within hydrogels to speed up degradation and better nurtured encapsulated cells. The degradation rate was further controlled by the ratio of modified PVAs and the inclusion of GelMA. Even cell distribution and high long-term encapsulated cell viability was observed over 2 weeks of culture. MSC markers were conserved during 2 weeks of encapsulation. MSC-containing hydrogel foams illustrated the ability to support cell migration in vitro and ex vivo. This hybrid hydrogel foam system demonstrates many versatile features that could enable its potential future use in irregularly shaped chronic wound treatment, including facile porous scaffold fabrication, degradabiliy, regular and rapid cell encapsulation, and promotion of wound healing.

What is claimed is:

1. A hydrogel, comprising:
an amount of thiolated poly(vinyl alcohol); and
an amount of methacrylated poly(vinyl alcohol) cross-linked with the amount of thiolated poly(vinyl alcohol).

2. The hydrogel of claim 1, wherein the amount of thiolated poly(vinyl alcohol) and the amount of methacrylated poly(vinyl alcohol) are present in a ratio selected from group consisting of 1:1, 1:2, 1:3, and 1:4 by volume.

3. The hydrogel of claim 1, wherein a plurality of cells are encapsulated in the hydrogel.

4. The hydrogel of claim 3, wherein the hydrogel has a plurality of pores.

5. The hydrogel of claim 3, wherein at least a portion of the plurality of pores range in size from 126 to 448 micrometers.

6. The hydrogel of claim 1, further comprising an amount of methacrylated gelatin cross-linked with the amount of thiolated poly(vinyl alcohol) and the amount of methacrylated poly(vinyl alcohol).

7. The hydrogel of claim 6, wherein the amount of thiolated poly(vinyl alcohol), the amount of methacrylated poly(vinyl alcohol), and the amount of methacrylated gelatin are present in a ratio of 1:4:2 by volume.

8. The hydrogel of claim 7, wherein a plurality of cells are encapsulated in the hydrogel.

9. The hydrogel of claim 8, wherein the hydrogel has a plurality of pores.

10. The hydrogel of claim 9, wherein at least a portion of the plurality of pores range in size from 126 to 448 micrometers.

11. A method of forming a hydrogel, comprising the steps of:
forming an amount of thiolated poly(vinyl alcohol) from an amount of poly(vinyl alcohol);
forming an amount of methacrylated poly(vinyl alcohol) from an amount of poly(vinyl alcohol); and
cross-linking the amount of thiolated poly(vinyl alcohol) with the amount of methacrylated poly(vinyl alcohol).

12. The method of claim 11, wherein the amount of thiolated poly(vinyl alcohol) and the amount of methacrylated poly(vinyl alcohol) are present in a ratio selected from group consisting of 1:1, 1:2, 1:3, and 1:4 by volume.

13. The method of claim 11, further comprising the step of encapsulating a plurality of cells.

14. The method of claim 13, further comprising the step of gas blowing during the step of cross-linking the amount of thiolated poly(vinyl alcohol) with the amount of methacrylated poly(vinyl alcohol).

15. The method of claim 14, wherein the hydrogel has a plurality of pores and at least a portion of the plurality of pores range in size from 126 to 448 micrometers.

16. The method of claim 11, further comprising the step of forming an amount of methacrylated gelatin from an amount of gelatin prior to the step of cross-linking the amount of thiolated poly(vinyl alcohol) with the amount of methacrylated poly(vinyl alcohol).

17. The method of claim 16, wherein step of cross-linking the amount of thiolated poly(vinyl alcohol) with the amount of methacrylated poly(vinyl alcohol) includes cross-linking with the amount of methacrylated gelatin.

18. The method of claim 17, wherein the amount of thiolated poly(vinyl alcohol), the amount of methacrylated poly(vinyl alcohol), and the amount of methacrylated gelatin are present in a ratio of 1:4:2 by volume.

19. The method of claim 18, further comprising the step of encapsulating a plurality of cells.

20. The method of claim 19, further comprising the step of gas blowing during the step of cross-linking the amount of thiolated poly(vinyl alcohol) with the amount of methacrylated poly(vinyl alcohol) and the amount of methacrylated gelatin.
